(19) United States
(12) Patent Application Publication
(10) Pub. No.: US 2022/0023229 A1
(43) Pub. Date: Jan. 27, 2022

(54) ATTACHMENT OF MEMBRANES FOR TRANSDERMAL DEVICES

(71) Applicant: DERMAL DIAGNOSTICS LTD, Loughborough (GB)

(72) Inventor: Dewan Fazlul Hoque Chowdhury, Loughborough (GB)

(21) Appl. No.: 17/312,237

(22) PCT Filed: Dec. 10, 2019
(86) PCT No.: PCT/EP2019/084517

§ 371 Date: Jun. 9, 2021

(30) Foreign Application Priority Data
Dec. 10, 2018 (GB) 1820080.8

(51) Int. Cl. A61K 9/70

(52) U.S. Cl. CPC A61K 9/7084

(57) ABSTRACT

An adhesive patch for securing a transdermal device to the skin comprises a double-sided adhesive layer. A permeable membrane element is located in an aperture in the adhesive layer. There is a small area of overlap where the adhesive layer adheres to the membrane element to secure it against movement during application and use of the patch.

### TECHNICAL FIELD

The invention relates to the field of transdermal devices for delivering drugs or obtaining samples through the skin of a human or animal subject. In particular, the invention relates to the secure location of a permeable membrane element that is intermediate between the device and the skin.

### BACKGROUND OF THE INVENTION

It is well known to use transdermal procedures for obtaining fluid samples or other analytes from a subject by withdrawal through the skin without the use of hypodermic needles. One example is the monitoring of glucose levels by persons who are diabetic. It is similarly known to use transdermal procedures to deliver drugs or other biologically active substances into the body of a subject. Electrochemical techniques such as iontophoresis or reverse iontophoresis may be used to enhance the transport of the substances or analytes in question across the skin.

It is desirable that the transdermal devices used to carry out such procedures should be capable of repeated use. Some elements such as a sensing electrode or a drug reservoir will need to be disposed of after each procedure but other elements such as the housing and control electronics are reusable. A double-sided adhesive patch is typically used to attach the transdermal device to the skin of each new subject, though the device may remain in place to carry out a series of procedures on the same subject. The patch must incorporate an element to provide an interface between the working area of the device (such as a set of electrodes) and the skin, across which the molecules of interest can be transported. A gel medium can be used but a fluid medium provides faster transport. Such a fluid must be held in the desired location and one solution is to provide a thin, permeable matrix, referred to herein as a membrane.

It is important to provide an air- and water-tight seal around the permeable membrane so that the fluid transport medium cannot leak out or transport the drug or analyte away from the working area of the device, which would reduce its efficiency. Similarly, the seal prevents the loss of moisture from the skin, which could change the operating conditions over the course of a series of measurements, making the results unreliable. The seal may be provided by the same adhesive layer that secures the patch to the skin.

For the correct, efficient and reliable operation of the transdermal device, it is important that the desired alignment is maintained between the permeable membrane element, the adhesive layer and the working area of the device.

### SUMMARY OF THE INVENTION

The invention provides an adhesive patch for a transdermal device according to claim 1.

Preferred but non-essential features of the invention are defined in the dependent claims.

The area of overlap between the membrane element and the adhesive layer is sufficient to secure the membrane element in its desired location within the aperture. This overcomes the risk that it could be displaced laterally during the application of the patch to the skin, resulting in faulty operation of the transdermal device. In the case of a transdermal sensor, the overlap also reduces relative movement between the membrane element and the sensing electrode during use, which can give rise to spurious signals.

In this specification, the term “underside” and cognate words are used to refer to the face of a patch or device that, in use, is closest to the skin of a subject. It will be understood that the patch and device may be used in any orientation, depending on the body part to which they are applied, and may similarly be manufactured, transported or stored in any orientation, without departing from the scope of the invention defined by the claims.

### THE DRAWINGS

FIG. 1 is a schematic plan, viewed from below, of an adhesive patch according to the invention, prior to use.

FIG. 2 is a schematic section on line A-A of FIG. 1.

FIG. 3 is a section, similar to FIG. 2, of an adhesive patch according to the invention during use.

FIG. 4 is a partial, schematic plan, viewed from below, of a second embodiment of the adhesive patch, with a differently shaped aperture.

FIG. 5 is a partial, schematic plan, viewed from below, of a third embodiment of the adhesive patch, with a differently shaped aperture.

FIG. 6 is a partial, schematic plan, viewed from below, of a fourth embodiment of the adhesive patch, with an elongated tab.

FIG. 7 is a schematic plan, viewed from below, of an adhesive patch according to a fifth embodiment of the invention.

FIG. 8 is a schematic section on line B-B of FIG. 7.

FIG. 9 is a partial, schematic plan, viewed from below, of a sixth embodiment of the adhesive patch, with a full-width tab.

In FIGS. 2, 3 and 7 the vertical scale and the spacing between layers are exaggerated to make the structure clearer.

At the heart of the patch is a membrane element 2, preferably formed from a disc of porous nylon. Other medically appropriate materials may be used. The membrane element 2 should be able to hold a fluid transport medium, such as a buffer solution. It should also permit the transport through the medium, across the thickness of the membrane, of molecules of an analyte that is to be sampled from the skin or molecules of a drug that is to be delivered to the skin.

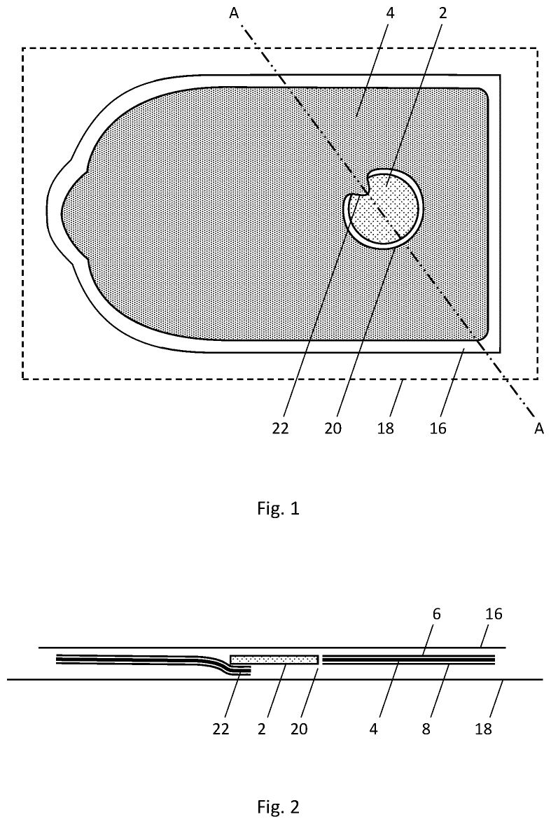

The patch also comprises an adhesive layer 4 that surrounds the membrane element 2. The upper surface 6 and the lower surface 8 of the adhesive layer 4 are coated with an adhesive that is suitable for medical use. Preferably, but not necessarily, the two coatings are of the same adhesive. The coating of the lower surface 8 should be suitable for releasably adhering the patch to the skin 10 of a subject. The coating of the upper surface 6 should be suitable for releasably adhering the patch to the underside of a transdermal device 12 and to the membrane element 2. Prior to use, the upper adhesive coating 6 may be protected by an upper removable liner 16 and the lower adhesive coating 8 may be protected by a lower removable liner 18. In FIG. 1, the lower liner 18 has been removed to reveal the other components but its outline is shown by dashed lines.

When adhering the patch to the underside of a transdermal device 12, the user normally rubs the lower removable liner 18 to expel air and ensure good contact between the upper adhesive coating 6 and the transdermal device 12 over the whole area of the patch. It has been found that, if the lower removable liner 18 is able to store static charge then, when the removable liner 18 is then peeled off, that charge is dissipated to the membrane 2 and hence to the electrode if the device 12 is a sensor. This leads to a very large initial sensor signal that can take up to 2 hours to dissipate and therefore prolongs the start-up time before the sensor can be used. One solution is to cover the external surface of the removable liner 18 with a polyurethane membrane (not illustrated), which prevents the build-up of static charge when the surface is rubbed.

The adhesive layer 4 is pierced by an aperture 20, in which the membrane element 2 is located. The aperture 20 substantially matches the size and shape of the membrane element 2 so that the adhesive layer 4 closely surrounds the membrane element except in a small area of overlap, where a tab 22 of the adhesive layer 4 overlaps the perimeter of the membrane element 2 and adheres to the underside of the membrane element. This adhesion by the tab 22 is sufficient to maintain the membrane element 2 in its desired location within the aperture 20, countering the risk that it could be displaced laterally during the application of the patch to the skin 10 or to the transdermal device 12. It further prevents the movement of the membrane in use, when the device has been applied to the skin. It has been noted that even very small movements of the membrane relative to the surface of a sensor (e.g. an enzymatic glucose oxidase based sensor) or sensor electrode leads to the generation of noise and erroneous and erratic signals. It is thought that these signals arise due to the physical perturbation of the surface of the electrode leading to the creation of amperometric noise signals. Noise signals have been observed that have often exceeded 5 multiples of the actual signal from the device due to the analyte. A further phenomenon has been observed whereby the erroneous signal has led to the re-setting of the baseline of the signal, thus rendering it impossible to remove the noise algorithmically. Similar errors have been noted if the membrane element 2 lifts away from the skin by even a few microns, for example if the patient twists an arm to which the patch is adhered. The anchoring of the membrane relative to both the skin and the electrode/sensor surface has therefore been demonstrated to be essential for the adequate functioning of such a system.

To apply the patch, it needs to be adhered to the transdermal device 12, having first replaced any disposable parts of the device such as a sampling chamber or drug reservoir. The transdermal device 12 comprises a working area on its lower surface, represented schematically in FIG. 3 by a set of electrodes 24 coupled to a controller 26. The upper removable liner 16 is unpeeled to expose the upper coating 6 of the adhesive layer 4, which is then adhered to the underside of the transdermal device 12 such that the membrane element 2 is aligned with the electrodes 24.

The adhesive patch is now used to adhere the transdermal device 12 to the skin 10. The lower removable liner 18 is unpeeled to expose the lower surface 8 of the adhesive layer 4 and the membrane element 2, which the tab 22 holds in place in the aperture 20 as the liner 18 is being removed. A drop of buffer solution or other fluid transport medium can then be applied manually to the membrane element 2 if required. Finally, the whole assembly is placed face down on a prepared area of the skin 10 and the transdermal procedure can begin.

The reader will understand that the illustrated embodiment is only one example of how the claimed invention may be put into practice. Naturally, the membrane element does not need to be circular: its size and shape may be varied to match the working area of the transdermal device with which the patch is intended to be used. Similarly, the overall size and shape of the patch, defined by the perimeter of the adhesive layer 2, may be varied to match a particular transdermal device.

The small area of overlap between the adhesive layer 4 and the membrane element 2 may be achieved in various ways other than the single tab 22 that is illustrated in the drawings. Two or more such tabs could be arranged around the perimeter of the membrane element 2 to provide additional stability. However, each tab reduces the area of contact between the membrane element 2 and the skin 10, thereby reducing the efficiency of the transdermal process. For this reason, it is preferred that fewer than four tabs are used and, in practice, one tab has been found to be sufficient.

The proportion of the surface area of the membrane element 2 that is occupied by the adhesive is critical to achieving an ideal balance between effective adhesion and effective transdermal transport. The overlapping area of the tab(s) preferably occupies less than 25% of the surface area of the membrane element and more preferably lies in the range 3% to 15%. The membrane element 2 should be in contact with the whole area of the electrode surface 24, thus the adhesive layer 22 should overlap the membrane on its skin-facing surface as indicated in FIG. 3, rather than lifting any of the edges of the membrane away from the electrode 24. This is required to ensure there is fluid communication and therefore adequate conductivity between the membrane element 2 and the whole surface of the electrode 24, without any differences in resistance in different regions of the membrane in contact with the electrode surface that might adversely affect the widely established function of a reference electrode, counter electrode and working electrode.

Furthermore, it is not possible to have an oversized membrane element 2, which might negate the need to have the adhesive layer 4 positioned between the membrane element 2 and the skin surface 10 and instead allow it to be positioned between the membrane element 2 and the transdermal device 12. The reason is that an oversized membrane will act to dilute the analyte that has been extracted from the skin and absorbed into the membrane, thus requiring sensors of lower limits of detection, which is very challenging given that this type of system will often be measuring pico-Molar quantities of an analyte such as glucose. The actual area of skin from which analyte will be extracted is usually small, and larger areas would potentially increase the possibility of skin irritation and other skin-related adverse events associated with adhesives being applied to the skin, as well as with scenarios where the skin is prepared using microprojection discs, for example, to perturb the top layer of the skin.

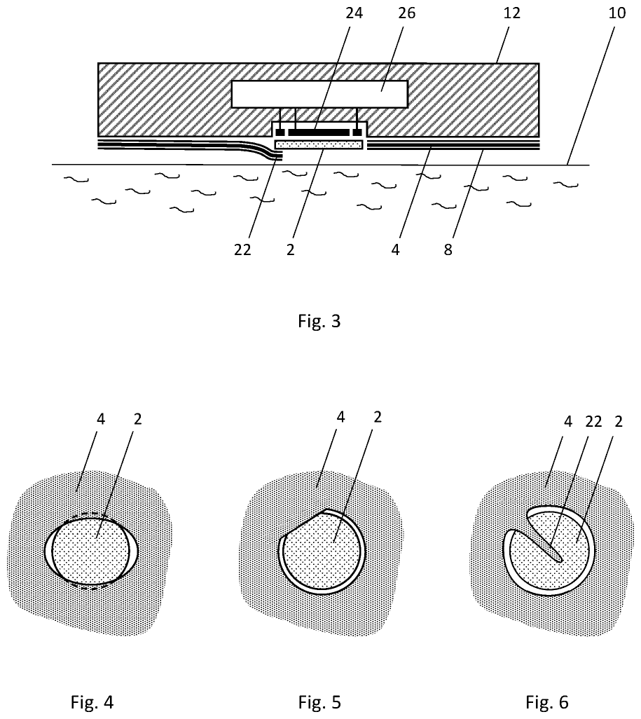

The use of one or more tabs 22 projecting from the edge of the aperture 20 of the adhesive layer minimizes the proportion of the perimeter of the membrane element 2 that is overlapped, for example to less than 20% of the length of the perimeter and preferably to only about 10% of the length. However, arrangements other than distinct tabs could also be used. For example, if the membrane element 2 is a disc, the aperture 20 could be made non-circular, having a portion of lower curvature (FIG. 4) or a straight chord (FIG. 5) that cuts across the perimeter of the disc. Compared with a tab, such an arrangement will result in a longer extent of overlap around the circumference of the disc but this can be offset by a smaller radial extent of overlap to keep the overall area of overlap reasonable.

It was mentioned above that lifting of the membrane element 2 away from the skin causes sensor errors. FIG. 6 illustrates one way to avoid this, namely, to extend the tab 22 of the adhesive layer 4 further into the aperture and preferably at least to the centre of the aperture. It is not excluded that the tab could extend fully across the aperture. The extended tab 22 provides adhesion between the membrane element 2 and the skin in the area where lifting is most likely to occur. This is at the expense of a reduced area of contact between the membrane element 2 and the skin so the extended tab should be made as narrow as possible, both to maintain a sufficient area of contact and to ensure that analyte from the skin can easily diffuse laterally through the membrane element 2 into the region that is obscured by the extended tab 22.

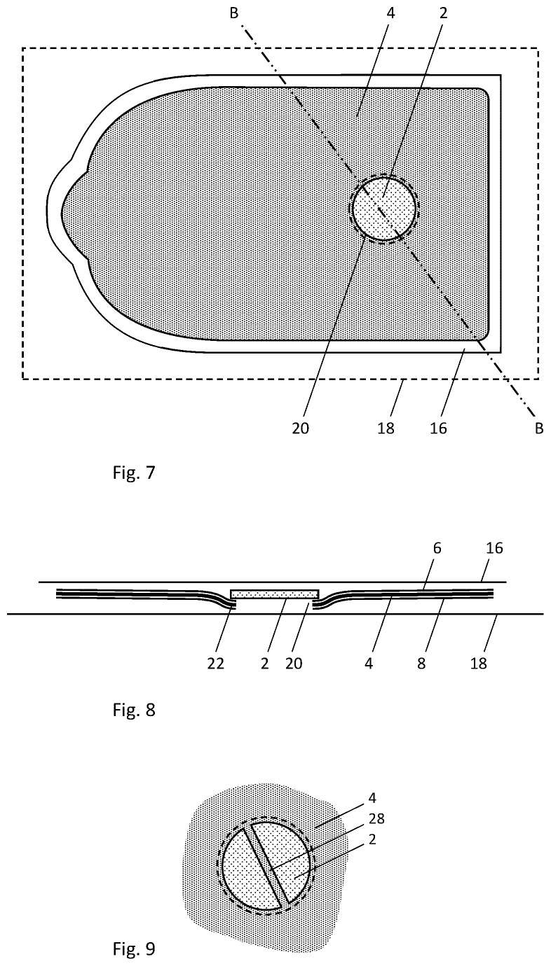

In an alternative arrangement, shown in FIGS. 6 and 7, the entire periphery of the membrane element 2 may be slightly overlapped by the adhesive layer 4, to anchor it to the skin 10 on one side and electrode surface 24 on the other side. However, this arrangement is less preferable because it has the potential to trap air between the membrane and the skin, which does not have the opportunity to escape around the periphery of the membrane element 2. Even small pockets of such trapped air have been found to compromise the performance of a sensor. This arrangement is also particularly prone to the problem of lifting of the membrane element 2 away from the skin, as previously described. This problem is reduced for smaller sizes of membrane in contact with the skin (i.e. within the perimeter of the overlapping adhesive layer 4). Generally a circular aperture of less than 15 mm in diameter, preferably less than 12 mm and more preferably less than 10 mm, will reduce the occurrence of such lifting. One could also employ the solution previously described, namely, a tab of the adhesive layer 4 that extends into or across the central region of the aperture 20. FIG. 9 illustrates an embodiment in which, instead of a tab extending from just one edge, the adhesive layer 4 comprises a narrow bar 28 that extends fully along a diameter of the aperture 20 to secure the centre of the membrane element 2 firmly to the skin.

1. An adhesive patch for a transdermal device, comprising:
an adhesive layer for releasably adhering the patch to the skin of a subject, opposite surfaces of the adhesive layer being coated with adhesive, the adhesive layer defining an aperture; and
a permeable membrane element located in the aperture in the adhesive layer;
wherein the adhesive layer surrounds the membrane element, except for a small area of overlap where the adhesive layer adheres to the membrane element.

2. A patch according to claim 1, wherein the area of overlap occupies less than 25% of the surface area of the membrane element.

3. A patch according to claim 2, wherein the area of overlap occupies between 3% and 15% of the surface area of the membrane element.

4. A patch according to any preceding claim, wherein the adhesive layer overlaps less than 20% of the perimeter of the membrane element.

5. A patch according to claim 4, wherein the adhesive layer overlaps less than 10% of the perimeter of the membrane element.

6. A patch according to claim 1, wherein the adhesive layer overlaps the membrane element at fewer than four positions around the perimeter of the membrane element.

7. A patch according to claim 6, wherein the adhesive layer overlaps the membrane element at only one position around the perimeter of the membrane element.

8. A patch according to claims 1, wherein the adhesive layer overlaps the membrane element continuously around the perimeter of the membrane element.

9. A patch according to claim 1, wherein the adhesive layer comprises a tab or bar that overlaps the membrane element and extends to the center of the membrane element.

10. A patch according to claim 1, wherein the membrane element is a disc.

11. A patch according to claim 1, wherein the membrane element is formed from porous nylon.

12. (canceled)

13. (canceled)

14. A patch according to claim 1, wherein the opposite surfaces of the adhesive layer comprise a first surface for releasably adhering the patch to the skin of the subject and a second surface, opposite to the first surface, that adheres to the membrane element in the area of overlap.

15. A patch according to claim 14, further comprising a removable liner that, prior to use of the patch, protects the first surface of the adhesive layer, the removable liner being formed from or covered with a material that does not store static charge when rubbed.

16. A patch according to claim 15, wherein the material is polyurethane.
